(19) United States
(12) Patent Application Publication
(10) Pub. No.: US 2006/0240490 A1
(43) Pub. Date: Oct. 26, 2006

(54) Methods and compositions for identifying target cell cytolytic lymphocytes in a sample

(76) Inventor: Peter P. Lee, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/396,349

(22) Filed: Mar. 30, 2006

Related U.S. Application Data
(63) Continuation of application No. PCT/US04/32278, filed on Oct. 1, 2004.
(60) Provisional application No. 60/508,709, filed on Oct. 2, 2003.
(60) Provisional application No. 60/530,798, filed on Dec. 17, 2003.

(51) Int. Cl. G01N 33/567; G01N 33/574

(52) U.S. Cl. 435/7.21; 435/7.23

(57) ABSTRACT

Methods and compositions for identifying target cell cytolytic lymphocytes, e.g., T-cells, such as neoplastic cell cytolytic T-cells, in a subject are provided. In practicing the subject methods, the sample is contacted with a target cell stimulator, e.g., a neoplastic cell, and a detectably labeled granule membrane protein specific binding agent. Following contact, any resultant labeled lymphocytes, e.g., T-cells, are identified as lymphocytes cytolytic for the target cell. Also provided are compositions, kits, and systems for practicing the subject methods. The subject methods find use in a variety of different applications, including disease/therapy monitoring applications and therapeutic applications.

### CROSS-REFERENCE TO RELATED APPLICATIONS

This application claims the priority benefit of U.S. provisional application Ser. No. 60/530,798, filed Dec. 17, 2003 and U.S. provisional application Ser. No. 60/508,709 filed Oct. 2, 2003; which applications are incorporated herein by reference in their entirety.

### GOVERNMENT RIGHTS

This invention was made with government support under federal grant no. NIH R01 CA 090809 awarded by the NIH. The United States Government may have certain rights in this invention.

### INTRODUCTION

### Background of the Invention

The ability to identify, enumerate, and viably isolate functional tumor-reactive lymphocytes, e.g., T cells, is vital to the success of immune monitoring and immunotherapy of cancer. A method for the isolation of viable T cells based on their functional capacity to kill target cells, particularly T cells reactive to tumor, would be extremely valuable in both research and clinical settings. Such a technique could be used to purify the rare, high-efficiency T cells capable of destroying tumor-antigen-bearing cells, expand them to high numbers, and reinfuse them for potential therapeutic benefit. Currently, methods exist which can enumerate and even isolate T cells based on their peptide-specificity (for example, recognizing tumor-antigen-bearing cells). However, most methods that measure functional capacity (particularly, cytolytic function) are either bulk assays that measure target killing and do not directly quantify effector cells, or do not allow viable separation of effector cells following the measurement.

As such, there is a need for the development of a method for identifying, enumerating, and viably isolating functional tumor-reactive lymphocytes, e.g., T cells.

### SUMMARY OF THE INVENTION

Methods and compositions for identifying target cell cytolytic lymphocytes, e.g., T-cells, such as neoplastic cell cytolytic T-cells, in a subject are provided. In practicing the subject methods, the sample is contacted with a target cell stimulator, e.g., a neoplastic cell, and a detectably labeled granule membrane protein specific binding agent. Following contact, any resultant labeled lymphocytes, e.g., T-cells, are identified as lymphocytes cytolytic for the target cell. Also provided are compositions, kits, and systems for practicing the subject methods. The subject methods find use in a variety of different applications, including disease/therapy monitoring applications and therapeutic applications.

### BRIEF DESCRIPTION OF THE DRAWINGS

FIG. 1. Tetramer+ clones express cytolytic granule proteins at high levels. Peripheral blood mononuclear cells from a healthy donor or the six clones from FIG. 1 were stained with G209-2M or MART26 tetramers, antibodies to CD8, granzyme A, granzyme B, and perforin, and analyzed by 4-color flow cytometry. (Top) Graphs are gated for CD8+ lymphocytes. Quadrants separating positive or negative expression of intracellular antigens were defined based on CD8− PBMC, few of which express these antigens. In PBMC (left), most CD8+ T cells express Granzyme A; a subset of these cells also express Granzyme B (bottom panels), and a subset of those also express Perforin (top panels). Two clones, one showing high tumor-cytolytic activity and one showing low activity are also shown. The clones have very high expression levels of the cytolytic granule proteins. (Bottom) The expression of tetramer-binding, CD8, and cytolytic granule proteins by these clones is quantified by the mean fluorescence intensity of the stained population. For comparison, the intensity of CD8+ PBMC from a healthy donor that express all three granular proteins (“CD8⁺gr⁺”) or none of these proteins (“CD8⁺gr⁻”) is shown.

FIG. 2. Cytotoxicity analyses of high and low recognition efficiency clones. (a) gp100-specific (476.104, 476.125, 476.101, 476.102) and (b) MART-specific (461.25, 461.29) CD8+ T cell clones were analyzed for their recognition efficiency for the native G209n or MART27 peptides by titration on T2 targets as described in materials and methods. CTL clones were combined with T2 cells at 10:1 effector to target ratio (10,000 targets per well) in triplicate wells for each measurement. Data is representative of two independent experiments. Data from a low recognition efficiency MART-specific clone (461.10) is included for comparison.

FIG. 3. Tetramer titration and dissociation analyses of high and low recognition efficiency clones. To assess the contribution of ‘structural avidity’ to differences in recognition efficiency, (a, b) gp100-specific (476.104, 476.125, 476.101, 476.102) and (c) MART-specific (461.25, 461.29) CD8+ T cell clones were stained with serial dilutions of pMHC tetramers made with either the native or heteroclitic peptide. To further analyze differences in TCR binding, rate of dissociation of bound pMHC tetramers from these clones was assessed upon competition with additional of an anti-HLA-A2 antibody (BB7.2). (d, e) gp100-specific (476.104, 476.125, 476.101, 476.102) and (f) MART-specific (461.25, 461.29) CD8+ T cell clones were stained with PMHC tetramers made with either the native or heteroclitic peptide at final concentrations to give MFI tetramer staining around 200. After collection of time 0, BB7.2 was added and samples were analyzed at the indicated timepoints. Data are then plotted as a fraction of staining at t=0. Data is representative of three independent experiments.

FIG. 4. CD107a functional assay using high and low recognition efficiency clones. (a) High and (b) low recognition efficiency clones were incubated with Malme-3M, mel526, and A375 then analyzed for CD107a mobilization by flow cytometric analysis. Cells were identified by forward and side scatter, then plotted for CD107a versus CD3 expression. Boxed populations indicate the percentage of cells staining positive for CD107a. (c) The relationship between CD107a mobilization and cytolytic activity of each clone are presented in a scatter plot. The graph shows that clones are segregated based on avidity and the r² value reflects a strong correlation.

FIG. 5. Identification of tumor-reactive T cells from a heterogeneous cell line by CD107a mobilization. (a) The cell line used was assessed for an increase in the gp100 specific population after stimulation with native peptide. Lymphocytes, identified by forward and side scatter, were gated for CD8+ cells, then plotted for CD8 versus tetramer staining. The number above the box represents the frequency of CD8+ cells that are G209n specific based on tetramer binding (left). The plot on the right is of the same cell line stained with a control A2/p53 264-272 tetramer. (b) The cell line was incubated with tumor targets. Lymphocytes, identified by forward and side scatter, were gated for CD8+ cells, then plotted for CD107a versus CD3 expression. These plots show that approximately 50% of cells mobilized CD107a in response to incubation with specific tumor targets (Malme-3M and mel526, but not A375). These values are consistent with tetramer staining data.

FIG. 6. Identification of high recognition efficiency, cytolytic T cells in post-melanoma vaccine PBMCs. (a) Tetramer analysis of three post-vaccine samples. Lymphocytes, identified by forward and side scatter, were gated for CD8+ cells, then plotted for CD8 versus tetramer staining. These plots show the vaccine induced CD8+ T cells that are G209n-specific (left) or G209-2M-specific (right). (b) These samples were incubated with Malme-3M, mel526, or A375 then analyzed for CD107a mobilization by flow cytometric analysis. Lymphocytes, identified by forward and side scatter, were gated for CD8+ cells, then plotted for CD107a versus CD3 expression. Boxed populations indicate the percentage of cells staining positive for CD107a. Small populations of CD8+, CD3+ cells in these patient samples mobilized CD107a in a specific manner, suggesting that these cells are tumor-reactive. (c) Cells were sorted based on CD107a mobilization from patient sample 10450.

FIG. 7. High recognition efficiency cytolytic T cells represent a small fraction of tetramer+ cells. Post-vaccine PBMC samples 10450, 10545, and 10356 were incubated with Malme-3M, mel526, or A375 then analyzed for both tetramer staining CD107a exposure by flow cytometric analysis. Lymphocytes, identified by forward and side scatter, were gated for CD8+ cells, then plotted for CD107a versus G209-2M tetramer staining. The cells were divided into four quadrants with the percentages of each quadrant indicated. Tetramer+ cells clearly segregated into CD107a+ and CD107a− subsets.

### FEATURES OF THE INVENTION

The subject invention provides method for assaying a sample for a cytolytic lymphocyte, e.g., T-cell, that is cytolytic for a target cell. In practicing the subject methods, the sample is combined with a target cell stimulator and a detectably labeled granule membrane protein (e.g., CD107a, CD107b, CD63, CTLA-4, Man-6-PR and/or TIA/GMP-17) specific binding agent. Any resultant lymphocytes, e.g., T-cells, labeled with the granule membrane protein specific binding agent are then identified as lymphocytes cytolytic for the target cell. In certain embodiments, the target cell is a neoplastic cell. In certain embodiments, the target cell stimulator is a cell (or derivative thereof) that endogenously expresses a target peptide of interest, e.g., a neoplastic cell or a virally infected cell. In certain embodiments, the sample is also contacted with detectably labeled lymphocyte, e.g., T-cell, specific binding agent, e.g., a detectably labeled CD3 specific binding agent. In certain embodiments, the sample is also contacted with a detectably labeled cytotoxic lymphocyte, e.g., T-cell, specific binding agent, e.g., a detectably labeled CD8 specific binding agent. In certain embodiments, the detectably labeled binding agent(s) are fluorescently labeled. In certain embodiments, lymphocytes labeled with the granule membrane protein specific binding agent are identified flow cytometrically. In certain embodiments, the method further includes separating any resultant lymphocytes labeled with the granule membrane protein specific binding agent from other components of the sample to produce a composition enriched for lymphocytes cytolytic for the target cell. In certain embodiments, the sample is a blood sample, e.g., a peripheral blood mononuclear cell sample. In certain embodiments, the sample is from a subject vaccinated with an immunogen for said target cell.

Also provided are methods of identifying the presence of a lymphocyte, e.g., T-cell, cytolytic for a target cell in a subject by assaying a sample from the subject for a cytolytic lymphocyte for the target cell, where the assay employed is as described above. In certain of these embodiments, the assay is performed at least two different times in order to monitor the subject for the presence of the lymphocyte cytolytic for the target cell, e.g., in methods of monitoring the subject for progression of a disease condition, such as a neoplastic disease condition.

Also provided are methods of treating a subject for a target cell mediated disease condition, e.g., a neoplastic condition, where the methods include obtaining a composition enriched for a population of lymphocytes, e.g., T-cells, cytolytic for the target cell using the protocols described above, and then expanding the population of lymphocytes, e.g., T-cells, in the composition followed by administration of the expanded population of lymphocytes, e.g., T-cells, to the subject.

Also provided is a substantially pure composition of viable lymphocytes, e.g., T-cells, cytolytic for a target cell, e.g., a neoplastic cell, where in certain embodiments, the lymphocytes are granule membrane protein positive. In certain embodiments, the lymphocytes are also CD8 positive. In certain embodiments, the composition is prepared according to the above-described methods.

Also provided are kits for use in practicing the subject methods, where the kits may include a detectably labeled specific binding agent that specifically binds to a granule membrane protein; and instructions for using the binding agent in the subject methods. In certain embodiments, the kits include a target cell stimulator, e.g., a cell, such as a neoplastic cell. In certain embodiments the kits include a detectably labeled lymphocyte, e.g., T-cell, specific binding agent, such as a detectably labeled T-cell specific binding agent that specifically binds to CD3. In certain embodiments the kits include a detectably labeled cytotoxic lymphocyte, e.g., T-cell, specific binding agent, such as a detectably labeled cytotoxic T-cell specific binding agent that specifically binds to CD8.

Also provided are systems for use in practicing the subject methods, where the systems include a detectably labeled granule membrane protein specific binding agent; a target cell stimulator; and a detector for said detectably labeled granule membrane protein binding agent.

Also provided are labeled samples that include a sample medium; a detectably labeled granule membrane protein specific binding agent; and a detectably labeled T-cell specific binding agent.

Also provided are sample loaded detection devices, e.g., a multiparameter flow cytometer devices, that include a fluid flow path loaded with a labeled sample of the subject invention.

### DETAILED DESCRIPTION OF THE INVENTION

Methods and compositions for identifying target cell cytolytic lymphocytes, e.g., T-cells such as neoplastic cell cytolytic T-cells, in a subject are provided. In practicing the subject methods, the sample is contacted with a target cell stimulator, e.g., a neoplastic cell, and a detectably labeled granule membrane protein specific binding agent. Following contact, any resultant labeled T-cells are identified as T-cells cytolytic for said target cell. Also provided are compositions, kits, and systems for practicing the subject methods. The subject methods find use in a variety of different applications, including disease/therapy monitoring applications and therapeutic applications.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods will be described first, followed by a review of representative applications in which the methods find use, as well as a review of representative kits and systems thereof that find use in practicing the subject methods.

Methods

As summarized above, the subject invention provides methods of identifying, and isolating, viable cytolytic lymphocytes, e.g., T-cells, in a sample. By “cytolytic lymphocyte” is meant a non-B lymphocyte that exhibits cytolytic activity, where cytolytic lymphocytes include, but are not limited to: cytolytic T-cells, natural killer (NK) cells, NKT cells and CD4⁺ T Cells which degranulate and kill target cells. While in the broadest sense the invention is directed to the identification of cytolytic lymphocytes as defined above, in many embodiments the methods and compositions of the invention are employed for the identification of cytolytic T-cells. Accordingly, for ease of further description of the invention, the invention will now be further described in terms of methods and compositions for use in the identification of cytolytic T-cells. However, the invention is not limited to the identification of cytolytic T-cells, but includes the identification of non-T-cell cytolytic lymphocytes, as described above.

By “cytolytic T-cell” is meant a cell that is cytotoxic for a target cell, i.e., a cell that is capable of killing a target cell, such as a neoplastic cell (e.g., a tumor cell), etc, such that that the T-cell is capable of killing a target cell, and is target cell reactive.

In practicing the subject methods, the following steps are typically practiced: 1) sample provision; 2) sample preparation/staining for granule membrane protein mobilization; 3) sample analysis; and 4) data analysis/processing. Each of these general steps is now described in greater detail.

Sample Preparation

In practicing the subject methods, the first step is to provide a sample that is to be assayed for the presence of the cytolytic T-cells of interest. The sample may be any of a variety of different types of samples, where the sample may be used directly from an initial source as is, e.g., where it is present in its initial source as a fluid, or preprocessed in some manner, e.g., to provide a fluid sample from an initial non-fluid source, e.g., solid; to dilute and or concentrate an initial fluid sample, etc.

As such, the first step of the subject methods is to obtain a suitable sample from the subject or patient of interest, i.e., a patient suspected of having or known to have the cytolytic T-cell of interest, such as a patient that is known to have the target cell for which the T-cell of interest is cytolytic. The sample may be derived from any initial source that would contain the cytolytic T-cells of interest (if present). Sample sources of interest include, but are not limited to, many different physiological sources, e.g. tissue derived samples, e.g. homogenates, and blood or derivatives thereof.

In many embodiments, the sample may be derived from fluids in which the T-cells of interest are at least suspected of being present. In many embodiments, a suitable initial source for the patient sample is blood. As such, the sample employed in the subject assays of these embodiments is generally a blood-derived sample. The blood-derived sample may be derived from whole blood or a fraction thereof, e.g. serum, plasma, etc., where in many embodiments the sample is derived from blood cells harvested from whole blood. Of particular interest as a sample source are mononuclear cells. As such, a preferred sample is one that is derived from peripheral blood mononuclear cells (PBMCs).

In these preferred embodiments in which the sample is a PBMC derived sample, the sample is generally a fluid PBMC derived sample. Any convenient methodology for producing a fluid PBMC sample may be employed. In many embodiments, the fluid PBMC derived sample is prepared by separating PBMCs from whole blood, i.e., collecting PBMCs, e.g., by centrifugation (such as by Ficoll-Hypaque density gradient centrifugation, where representative protocols for such separation procedures are disclosed in WO 98/15646 and U.S. Pat. No. 5,985,565; the disclosure of the latter of which is herein incorporated by reference.

The sample may be obtained from a variety of different subjects/patients/hosts. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Sample Preparation/Staining for Granule Membrane Protein Mobilization

Following provision of the fluid sample, the sample is labeled or stained with fluorescent labeling reagents for at least one granule membrane protein mobilization. Granule membrane proteins of interest include, but are not limited to: CD107a (also known as LAMP-1), CD107b (also known as LAMP-2), CD63, CTLA-4, Man-6-PR, and TIA/GMP-17). In certain embodiments, the granule membrane protein of interest is CD107a.

The sample is labeled or stained in a manner that detectably labels the specific granule membrane protein molecules of interest on the surface of T-cells that have mobilized to the surface of T-cells in response to the presence of a target cell stimulator. In this step of the subject invention, the sample to be assayed, e.g., the PBMC fluid sample, is combined with a detectably labeled granule membrane protein, e.g., CD107a, specific binding agent and a target cell stimulator to produce a reaction mixture, and the reaction mixture is maintained under conditions sufficient for granule membrane protein, e.g., CD107a, molecules to mobilized to the surface T-cells present in reaction mixture that are cytolytic for the target cell of interest.

Combination of the sample with the granule membrane protein, e.g., CD107a, specific binding agent and target cell stimulator is achieved by contacting the sample with the granule membrane protein, e.g., CD107a, specific binding agent and the target cell stimulator. Contact of the sample with the granule membrane protein, e.g., CD107a, specific binding agent and the target cell stimulator is achieved using any convenient protocol. As such, in certain instances the granule membrane protein, e.g., CD107a, specific binding agent and target cell stimulator is introduced into the sample. In yet other instances, the sample is introduced into a container that includes the granule membrane protein, e.g., CD107a, specific binding agent and the target cell stimulator, e.g., a container that may include both of the granule membrane protein, e.g., CD107a, specific binding agent and the target cell stimulator, as described in greater detail below. Other protocols may also be employed, so long as the sample and granule membrane protein, e.g., CD107a, specific binding agent/target cell stimulator are contacted under conditions such that the label may bind to granule membrane protein, e.g., CD107a, on the surface of T-cells cytolytic for the target cell of interest, if such cells are present in the sample.

The granule membrane protein, e.g., CD107a, specific binding agent may be any convenient binding agent that specifically binds to the granule membrane protein, e.g., CD107a, when present on the T-cell surface.

As indicated above, in certain embodiments the granule membrane protein of interest is CD107a. As is known in the art, CD107a is a type I membrane glycoprotein found on the surface of a number of distinct cell types, including T-cells. The nucleic acid coding sequence and amino acid sequence of the human protein is deposited in Genbank and has an accession no. of J04182, and is also reported in Fukuda et al., J. Biol. Chem. (1988) 263: 18920-18928; the nucleic acid coding sequence and amino acid sequence of the mouse protein is deposited in Genbank and has an accession no. of J03881 and M32015, and is also reported in Chen et al., J. Biol. Chem. (1988) 263:8754-8758; and the nucleic acid coding sequence and amino acid sequence of the rat protein is deposited in Genbank and has an accession no. of M34959, and is also reported in Howe et al., Proc. Nat'l Acad. Sci USA (1988) 85:7577-7581.

A feature of the CD107a binding agent employed in the subject methods is that it specifically binds to CD107a, and does not substantially bind to other cellular entities that may be present on the cell, such as other proteins found on the surface of T-cells. As such, the CD107a binding agent employed typically shows minimal, if any, cross-reactivity with other cell surface proteins present on T-cells or other cells in the sample.

In the broadest sense, the granule membrane protein, e.g., CD107a, binding agent may be labeled with any of a number of different types of labeling agents, where the labeling agents may be part of signal producing system made up of one or more components, where labeling component that binds to the granule membrane protein, e.g., CD107a, may be directly or indirectly detectable. Examples of labels that permit direct measurement include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In many embodiments of interest, the granule membrane protein, e.g., CD107a, binding agent is a fluorescent labeling reagent. The granule membrane protein, e.g., CD107a, fluorescent labeling reagent may be a variety of different types of reagents. In many embodiments, the reagent is a fluorescently labeled member of a specific binding pair, where granule membrane protein, e.g., CD107a, present on the surface of the cellular analyte is typically the other member of the specific binding pair. While a variety of types of agents may serve as a specific binding pair member, including peptides, aptamers, lectins, antibiotics, substrates, and the like, in many embodiments, the specific binding pair member is an antibody or binding fragment/mimetic thereof, e.g., scFv, FAB, etc (hereinafter collectively referred to as an “antibody ligand”). The specific binding pair, e.g., antibody ligand, may be labeled with a variety of different fluorescent labels, including, but not limited to: phycoerythrin (“PE”), fluorescein isothiocyanate (“FITC”), allophycocyanin (“APC”), Texas Red (“TR”, Molecular Probes, Inc.), peridinin chlorophyll complex (“PerCp”), CY5 (Biological Detection System) and conjugates thereof coupled to PE (e.g., PE/CY5 (CyChrome), PE/APC and PE/TR); etc. Where the specific binding pair member is an antibody ligand, the ligand can be directly conjugated to a fluorescent label or can be indirectly labeled with, for example, a goat anti-mouse antibody conjugated directly to the fluorescent label. Direct conjugation is found, however, in many embodiments.

As indicated above, also combined with the sample in this step of the subject methods is a target cell stimulator. The term “target cell stimulator” is used to describe an entity that acts to stimulate a T-cell so that, if it is cytolytic towards the target cell of interest, it mobilizes the granule membrane protein, e.g., CD107a, of interest. In the broadest sense, the target cell stimulator may be any entity or composition that is capable of causing this desired response in T-cells of interest. In many embodiments, the target cell stimulator is a cell or derivative thereof which has the T-cell stimulatory activity of the target cell of interest, where the cell may be the specific target cell of interest or a different type of cell that nonetheless causes the desired T-cell response. A feature of many embodiments of the subject invention is that the target cell stimulator, or derivative thereof, is one that endogenously expresses the target peptide that is recognized by the T-cell and characterizes the target cell. As such, the target cell stimulator is not an “artificial” target cell that has been pulsed with the target peptide of interest, but instead is one that endogenously expresses the target peptide such that the target peptide is present and produced in amounts found in the target cell. In certain embodiments, the target cell stimulator is a neoplastic cell, where neoplastic cells of interest include those types of neoplastic cells specifically listed below. In certain embodiments, the target cell stimulator is a virally infected cell. In yet other embodiments, the target cell stimulator may be a non-cellular composition that acts like the target cell to cause the desired granule membrane protein, e.g., CD107a, mobilization in cytolytic T-cells, where representative non-cellular compositions of interest may include a lysate of the above representative cellular target cell stimulators, and the like.

In addition to the above components, the sample may also be combined with one or more additional labeling reagents intended to label one or more additional markers on the surface of the T-cells of interest at least suspected of being in the assayed sample. As the sample is may be contacted with at least one additional specific label reagent, the sample may be contacted with one or more distinct types specific labels, depending on the number of different additional cell markers for which the sample is to be assayed. As such, the number of different additional specific labels that is contacted with the sample may be 1 or more, 2 or more, 4 or more, 6 or more, where in certain embodiments, the number ranges from about 1 to 5, often from about 1 to 4 and more often from about 1 to 3. Any two specific label reagents are considered different if they specifically bind to different cellular markers. As with the granule membrane protein specific binding agent, the at least one additional labeling reagent may be labeled with a variety of different types of types of labels, including both indirectly and directly detectable labels. As with the granule membrane protein specific binding agent, the one or more additional specific reagents are, in many embodiments, fluorescently labeled members of a specific binding pair, where a cell surface marker, e.g., ligand present on the surface of the cell, is typically the other member of the specific binding pair. As indicated above, while a variety of types of agents may serve as a specific binding pair member, including peptides, aptamers, lectins, antibiotics, substrates, and the like, in many embodiments, the specific binding pair member is an antibody or binding fragment/mimetic thereof, e.g., scFv, FAB, etc (hereinafter collectively referred to as an “antibody ligand”). As described above, the specific binding pair, e.g., antibody ligand, may be labeled with a variety of different fluorescent labels, including, but not limited to: phycoerythrin (“PE”), fluorescein isothiocyanate (“FITC”), allophycocyanin (“APC”), Texas Red (“TR”, Molecular Probes, Inc.), peridinin chlorophyll complex (“PerCp”), CY5 (Biological Detection System) and conjugates thereof coupled to PE (e.g., PE/CY5 (CyChrome), PE/APC and PE/TR); etc. Where the specific binding pair member is an antibody ligand, the ligand can be directly conjugated to a fluorescent label or can be indirectly labeled with, for example, a goat anti-mouse antibody conjugated directly to the fluorescent label. Direct conjugation is preferred, however, in many embodiments.

While the specific nature of the one or more additional specific binding reagents used to label or stain the sample may vary depending on the nature of the assay and the method of detection of the T-cells of interest, in many embodiments the additional labels are ones that aid is distinguishing T-cells from non-T-cells in the sample. Representative cell surface markers that may labeled with specific binding agents for this purpose include, but are not limited to: CD8 (found on cytotoxic T-cells), CD3 (found on T-cells), CD19 (found on B-lineage cells (e.g., for distinguishing such cells from T-cells), and the like.

In addition to the above components, where desired the sample may also be labeled or stained with a label that specifically binds to a particular T-cell antigen receptor. For example, the sample may be stained or labeled with a multimeric binding complex that includes major histocompatibility complex protein subunits having a homogeneous population of peptides bound in the antigen presentation site, e.g., a peptide/MHC tetramer label, where such labels (as well as the preparation and use thereof are known in the art in the described in U.S. Pat. No. 5,635,363; the disclosure of which is herein incorporated by reference. In such embodiments, the peptide component of the subject multimeric labeling agents is typically a peptide specifically associated with the target cell for which the T-cells of interest are cytotoxic.

In certain embodiments, in addition to the combining the sample with labeling/staining agents as outlined above, a calibration standard may be added to the sample in order to obtain the absolute count of the labeled cells identified in the sample. The microparticle used as a calibration standard is made of a material that avoids clumping or aggregation, and is typically labeled, e.g., fluorescent. Fluorescence can be achieved by selecting the material that comprises the microparticle to be autofluorescent or it can be made fluorescent by being tagged with a fluorescent dye to appear autofluorescent. The fluorescence of the microparticles may be such that in one fluorescence channel it is sufficiently greater than noise from background so as to be distinguishable and also, in at least certain embodiments, must be distinguishable in other fluorescence channel(s) from the fluorescent dye(s) used as part of the analyte specific fluorescence marker(s). One log difference between the dye(s) and the microparticle fluorescence is sufficient. Microparticles having these properties may be selected from the group consisting of fixed chicken red blood cells, coumarin beads, liposomes containing a fluorescent dye, fluorescein beads, rhodamine beads, fixed fluorescent cells, fluorescent cell nuclei, microorganisms and other beads tagged with a fluorescent dye. The concentration of the microparticle should be greater than or equal to the number of cells to be counted. Generally, a 3:1 ratio of beads to cells is sufficient, although a 1:1 ratio is preferred. A variety of such calibration beads and protocols for their use in obtaining absolute cell counts via flow cytometry are known and commercially available, where representative calibration products include, but are not limited to: the TruCOUNT™ bead fluorescent product sold by Becton Dickinson; and the like. Instead of using such a calibration product, absolute counts may be obtained using alternative protocols, e.g., spiking in a counted liquid bead suspension; driving the sample through the instrument by syringe or other metered positive displacement means; etc.

Contact of the sample with the labeling reagents, including optional labeling reagents described above, is performed under incubation conditions that provide for binding of labeling reagents to their respective cell surface markers, if present, in the sample. The labeling reagents and samples may be contacted at any convenient temperature, e.g., room temperature or a temperature ±15, e.g., ±10° C. The amount of the different reagents that are contacted may vary and optimum amounts can readily be determined empirically, where representative amounts of different reagents such as effector/target cell ratio and CD107a specific antibody amounts are provided in the Experimental Section, below. Contact typically is performed with mixing or agitation, e.g., with vortexing etc., to provide for sufficient combination of the labeling reagents and the sample. The sample is then typically maintained or incubated for a period of time prior to flow cytometric analysis, as is known in the art.

Following the above incubation step, the sample may be assayed immediately or stored for assay at a later time. If stored, in many embodiments the sample is stored at a reduced temperature, e.g., on ice.

Sample Analysis/Detection of Cytolytic T-Cells

Once the sample has been prepared as described above by combining the sample with the granule membrane protein, e.g., CD107a, specific binding agent an target cell stimulator (as well as any desired additional reagents as described above), the sample is then analyzed to detect the presence of T-cells labeled with the granule membrane protein, e.g., CD107a, binding agent and thereby identify cytolytic T-cells in the sample.

The particular analysis/label detection protocol employed in this step of the subject methods may vary depending on the nature of the different labeling agents employed to stain the sample. Where the labeling agents employed in the methods are fluorescent labeling agents, such as the representative fluorescent labeling reagents described above, the sample may conveniently be flow cytometrically analyzed to flow cytometrically detect the presence of, either qualitatively or quantitatively, the cytolytic T-cells present in the sample.

The amount of sample that is assayed may vary depending on the particular application in which the method is practiced, and may range from about 10^{e4} PBMC to about 10^{e8} PBMC, usually from about 10^{e5} PBMC to about 10^{e6} PBMC.

Flow cytometry is a well-known methodology using multi-parameter data for identifying and distinguishing between different cell/particle types in a sample. In flow cytometrically analyzing the sample prepared as described above, the sample is first introduced into the flow path of the flow cytometer. Generally, the sample is analyzed by means of flow cytometry wherein the cells present in a flow path of a flow cytometer device are passed substantially one at a time through one or more sensing regions (wherein each of the cells is exposed separately individually to a source of light at a single wavelength and measurements of typically at least two light scatter parameters and measurements of one or more fluorescent emissions are separately recorded for each cell), and the data recorded for each cell is analyzed in real time or stored in a data storage and analysis means, such as a computer. U.S. Pat. No. 4,284,412 describes the configuration and use of a typical flow cytometer equipped with a single light source while U.S. Pat. No. 4,727,020 describes the configuration and use of a flow cytometer equipped with two light sources. The disclosures of these patents are herein incorporated by reference.

More specifically, in a flow cytometer, cells are passed, in suspension, substantially one at a time in a flow path through one or more sensing regions where in each region each cell is illuminated by an energy source. The energy source generally comprises an illumination means that emits light of a single wavelength such as that provided by a laser (e.g., He/Ne or argon) or a mercury arc lamp with appropriate filters. Light at 488 nm is a generally used wavelength of emission in a flow cytometer having a single sensing region. For flow cytometers that emit light at two distinct wavelengths, additional wavelengths of emission light that are commonly employed include, but are not limited to: 535 nm; 635 nm; 610 nm; 660 nm;

780 nm; and the like.

In series with a sensing region, multiple light collection means, such as photomultiplier tubes (or “PMT”), are used to record light that passes through each cell (generally referred to as forward light scatter), light that is reflected orthogonal to the direction of the flow of the cells through the sensing region (generally referred to as orthogonal or side light scatter) and fluorescent light emitted from the cell, if it is labeled with fluorescent marker(s), as the cell passes through the sensing region and is illuminated by the energy source. Each of forward light scatter (or FSC), orthogonal light scatter (SSC), and fluorescence emissions (FL1, FL2, etc.) comprise a separate parameter for each cell (or each “event”). Thus, for example, two, three or four parameters can be collected (and recorded) from a cell labeled with two different fluorescence markers.

Flow cytometers further include data acquisition, analysis and recording means, such as a computer, wherein multiple data channels record data from each PMT for the light scatter and fluorescence emitted by each cell as it passes through the sensing region. The purpose of the analysis system is to classify and count cells wherein each cell presents itself as a set of digitized parameter values.

Data Analysis/Processing

In analyzing the sample for the cytolytic T-cells of interest, the flow cytometer may be set to trigger on a selected parameter in order to distinguish the T-cells of interest from background and noise. “Trigger” refers to a preset threshold for detection of a parameter. It is typically used as a means for detecting passage of a cell or other particle through the laser beam. Detection of an event that exceeds the threshold for the selected parameter triggers acquisition of light scatter and fluorescence data for the particle. Data is not acquired for cells or particles that cause a response below the threshold. The trigger parameter may be the detection of forward scattered light caused by passage of a cell or particle through the light beam. The flow cytometer then detects and collects the light scatter and fluorescence data for the cell or bead.

A particular subpopulation of interest is then further analyzed by “gating” based on the data collected for the entire population. To select an appropriate gate, the data is plotted so as to obtain the best separation of subpopulations possible. This procedure is typically done by plotting forward light scatter (FSC) vs. side (i.e., orthogonal) light scatter (SSC) on a two-dimensional dot plot. The flow cytometer operator then selects the desired subpopulation of cells (i.e., those cells within the gate) and excludes cells that are not within the gate. Typically, the operator selects the gate by drawing a line around the desired subpopulation using a cursor on a computer screen. Only those cells within the gate are then further analyzed by plotting the other parameters for these cells, such as fluorescence.

Flow cytometric analysis of the sample, as described above, yields qualitative and quantitative information about the presence of the cytolytic T-cells of interest in the sample being assayed. In many embodiments, the above analysis yields counts in the sample.

Using the above methods, one can obtain highly sensitive readings the presence and amount of cytolytic T-cells in a sample. Generally, achievable sensitivity for cellular analytes is at least about 1 in 100 CD8+ T cells, typically at least about 1 in 1,000 CD8+ T cells and often at least about 1 in 10,000 CD8+ T cells, with a detection limit in many embodiments of from about 0.1 to 10 cells per ml.

In certain embodiments, the methods may be methods of not just identifying the presence of cytolytic T-cells in a sample, by separating the identified cytolytic T-cells from other constituents of the sample. The cytolytic T-cells of interest may be separated from a complex mixture of cells, e.g., as may make up the other constituents of the sample, by techniques that enrich for cells having the above characteristics.

Knowing the identifying surface marker population of the T-cells of interest, separation of the T-cell populations may use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, eg. plate, or other convenient technique. Techniques providing accurate separation include fluorescence activated cell sorters (as described above in connection with identification protocols), which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the selected cells.

As indicated above, the affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

As indicated above, of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then separated as to the presence of cell surface markers that identify the target T-cell populations of interest, e.g., the presence of CD107a, CD8, CD3 and antigen specific receptor, such as tumor cell antigen specific receptor, as exemplified in the experimental section below.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for cytolytic T-cells of interest may be achieved in this manner. The subject population will be at or about 90% or more of the cell composition, and preferably be at or about 95% or more of the cell composition. The desired cells are identified by their surface phenotype, by the ability to kill target cells for which they are cytolytic, e.g., neoplastic/tumor cells, and having a high recognition efficiency for the target cells for which they are cytolytic. The enriched cell population may be used immediately, or may be frozen at liquid nitrogen temperatures and stored for long periods of time, being thawed and capable of being reused. The cells will usually be stored in 10% DMSO, 50% FCS, 40% RPMI 1640 medium. Once thawed, the cells may be expanded by use of growth factors or stromal cells associated with hematopoietic cell proliferation and differentiation.

The enriched cell population may be grown in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin.

As such, the above-described methods provide ways of identifying the presence of cytolytic T-cells in a sample, and also ways of preparing compositions enriched for a cytolytic T-cells from a sample. In many embodiments, the methods are methods of identifying cytolytic T-cells for a specific type of target cell in sample, as well as methods of isolating such cytolytic T-cells from the sample, e.g., in a manner that maintains the viability of the isolated T-cells.

The methods may be employed to isolate T-cells that are cytolytic, i.e., capable of killing or cytotoxic for, a wide variety of different types of target cells. Target cells of interest include, but are not limited to disease causing cells, e.g., hazardous/pathogenic cellular microorganisms, such as Pneumococcus, Staphylococcus, Bacillus. Streptococcus, Meningococcus, Gonococcus, Eschericia, Klebsiella, Proteus, Pseudomonas, Salmonella, Shigella, Hemophilus, Yersinia, Listeria, Corynebacterium, Vibrio, Clostridia, Chlamydia, Mycobacterium, Helicobacter and Treponema; protozoan pathogens, and the like; as well as disease causing cells endogenous to the host, e.g., neoplastic cells, including cancerous cells. Specific representative neoplastic target cells include those found in the following representative types of cancers: carcinomas, melanomas, sarcomas, lymphomas and leukemias, etc.

Utility

The subject methods find use in a variety of different applications where one wishes to identify, and/or isolate, cytolytic lymphocytes, e.g., T-cells. One representative application in which the subject methods find use is monitoring the progression of a target cell mediated disease condition, e.g., by using the subject methods to monitor the population of target cell specific cytolytic T-cells over a period of time and using the obtained data to evaluate the progress of the disease condition, e.g., whether the condition is getting worse or better, how a particular treatment regimen is progressing, etc. In such applications, a sample from the host is typically assayed at least two different times so as to monitor the population of the T-cells of interest over the time frame characterized by the at least two different times, where the number of times in which a sample is assayed will necessarily vary depending on the particular monitoring protocol. In certain embodiments, the host that is monitored is one that has been vaccinated for the target cell of interest, e.g., with an immunogen specific for the target cell for which the identification of cytolytic T-cells is desired.

In another representative application, the subject methods are employed in therapeutic protocols per se in order to produce therapeutic agents, i.e., therapeutic cytolytic T-cells. In such applications, the methods are employed to produce an enriched cytolytic T-cell composition from an initial sample of the subject to be treated. The enriched isolated T-cell composition may then be expanded ex vivo to produce an increased population of cytolytic T-cells. In certain embodiments, a feature of the subject methods is that the harvested population of cells is expanded, where the expansion step occurs at some point in time prior to reintroduction of the cells to the subject of origin. In the expansion step, the number of T-cells in the harvested cell collection is increased, e.g., by at least about 4 fold, such as by at least about 4 fold as compared to the originally isolated amount, such that at least in certain embodiments the final number may be from about 100- to about 100,000-fold or more greater than the original number of cells. As such, the isolated cells are proliferated to produce an expanded population of harvested T-cells.

The isolated cells may be proliferated in this step according to any convenient protocol. For example, the cells are proliferated or enhanced by contacting the cells with an expansion agent, by which is meant an agent that increases the number of cells by causing cellular proliferation. A variety of different such agents are known, where representative agents include, but are not limited to: growth factors, accessory cells, ligands of specific activation receptors that may be monoclonal antibodies or antigens, and the like. One representative such protocol is described in U.S. Pat. No. 6,352,694; the disclosure of which is herein incorporated by reference.

Following isolation and expansion of the cytolytic target cells, an effective amount of the expanded population of cells is reintroduced to the host, e.g., by reinfusion or other convenient administration protocol. By effective amount is meant an amount effective to achieve the desired treatment of the host. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. In certain embodiments, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits

In yet another aspect, the present invention provides kits for practicing the subject methods, e.g., for flow cytometrically assaying a sample for cytolytic T-cells, for isolating cytolytic T-cells from a sample, etc. The subject kits at least include a granule membrane protein, e.g., CD107a, specific binding agent. In addition, the kits may include a number of additional components, e.g., additional marker labeling agents/stains, calibration beads, target cell stimulators, etc., as described above. In addition, the kit may include one or more additional compositions that are employed, including but not limited to: buffers, diluents etc., which may be required to produce a fluid sample from an initial non fluid, e.g., solid sample, or to otherwise prepare an initial fluid sample for analysis, e.g., enrich or dilute a sample with respect to the analytes of interest.

The above components may be present in separate containers or one or more components may be combined into a single container, e.g., a glass or plastic vial. For example, in certain embodiments are kits that include a single container that includes at least the calibration beads, when present, and serves as a sample preparation container, e.g., into which sample may be added as well as labeling reagents. In certain embodiments, the labeling reagents may also be present in the container such that a single container contains all necessary reagents and one need just add sample to the container in order to prepare and label the sample for flow cytometric analysis.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems for use in practicing the subject methods. The subject systems include the various reagent components required to perform the assay, e.g., the cellular and non-cellular labeling reagents, as well as label detector, e.g., a flow cytometric detector. Representative flow cytometric devices include, but are not limited, to those devices described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of which are herein incorporated by reference.

The following examples are offered by way of illustration and not by way of limitation.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

I. Methods

A. Generation of T Cell Clones:

CD8+ T cell clones were derived from PBMC samples of melanoma patients after vaccination with the heteroclitic peptides MART 26-35 (A26L) and gp100 209-217 (210M) in incomplete Freund's adjuvant (IFA) at the USC Norris Cancer Center, Los Angeles, Calif. under an IRB approved protocol. PBMC samples were analyzed for TAA-specific T cells using HLA-A*0201/peptide tetramers made with MART A26L, MART 27-35 (native), gp100 210M, and gp100 209-217 (native). Cells were stained and analyzed by FACS as previously described (Lee, P. P. et al. Characterization of circulating T cells specific for tumor-associated antigens in melanoma patients. Nature Medicine 5, 677-85 (1999)). CD8+ tetramer+ T cells were sorted, one cell per well containing 100 μl of CTL media (Iscove's Modified Dulbecco's Medium, IMDM, with 10% FBS, 2% human AB sera, and Penicillin, Streptomycin, and L-Glutamine) supplemented with 100 U/ml IL-2, under sterile conditions into 96 well plates using a FACS Vantage (Becton Dickinson, San Jose, Calif.). Sorted cells were expanded in vitro using standard protocols. Briefly, irradiated feeder cells (JY cells and fresh PBMCs) were added to wells containing the sorted T cells and the 96 well plates were incubated at 37° C., 7% CO₂. Potential clones become visible around day 14 and were then transferred to 24 well plates containing 1 ml CTL media with 100 U/ml IL-2. Wells were selected based on cell confluency for expansion and further analysis. Clones confirmed to be tetramer+ were expanded in T-25 flasks containing irradiated JY cells and fresh PBMCs in 25 ml CTL media containing PHA. IL-2 was added to a final concentration of 50 U/ml on day 1 and then every 2 days thereafter for 2 weeks. CD8+ T cell clones were also generated based on CD107a expression using identical methodology.

B. Generation of T Cell Lines:

PBMC from a post-vaccine patient with a 0.8% gp100 tetramer-specific T cell population were stimulated with T2 cells pulsed with gp100 209-217 (native, G209n) peptide at 2 μg/ml. Briefly, T2 cells were pulsed in a 15 ml conical tube for one hour at 37° C. and then irradiated at 12,000 rads. T2 cells were washed and 1.6×10⁶ cells were added to 10⁶ ficoll-purified PBMCs in 1 ml CTL media in a 24 well plate. IL-2 was added the following day at a final concentration of 100 U/ml. Cells were stimulated approximately every 2 weeks depending on growth. The second and third stimulations were done in T-25 and T-75 flasks, respectively, to obtain as many G209n specific T cells as possible. The expansion protocols were scaled up according to the surface area of the bottom of the flasks relative to a well in the 24-well plate. After 3 stimulations, the cell line count was over 10⁸ with the G209n specific T cell population representing over 50% of CD8+ cells. Cells were frozen at 10⁷ cells/vial and analyzed for pMHC tetramer binding by flow cytometry the same day they were used in the CD107 mobilization assay.

C. Determination of Recognition Efficiency:

Chromium-labeled T2 targets were pulsed with a range of peptide concentrations, generally starting at 10⁻⁶ M and decreasing by log steps to 10⁻¹⁴ M. T cell clones were incubated with T2 targets at 10:1 E:T ratios for 4 hours, then chromium release was measured and percentage cytotoxicity calculated by standard methods. Prior to each cytotoxicity assay, clones underwent ficoll-hypaque centrifugation to remove dead feeder cells, and were determined to be >80% CD8+ tetramer+ T cells by FACS. The E:T ratio was based upon live T and target cells. For each T cell clone, % cytotoxicity was plotted against peptide concentration. The peptide concentration at which the curve crosses 50% cytotoxicity was defined as the recognition efficiency of that clone (Margulies, D. H. TCR avidity: it's not how strong you make it, it's how you make it strong. Nat Immunol 2, 669-70 (2001)) and rounded to the nearest log.

D. CD107 Mobilization Assay

1. Target Cells:

The HLA-A*0201+ melanoma lines Malme-3M and A375 were purchased from ATCC and maintained according to their instructions. The HLA-A*0201+ melanoma line mel526 was a kind gift from Dr. Cassian Yee (Fred Hutchinson Cancer Center, Seattle, Wash.). While Malme-3M and mel526 express both MART and gp100, A375 does not express MART or gp100 and served as a negative control. Expression (or lack of) of these antigens by each cell line was further confirmed by immunohistochemical staining. These cells adhere to plastic and were trypsinized using Trypsin/EDTA solution (Gibco) before use. They were washed and resuspended to the appropriate concentration (usually 10⁷/ml) in CTL media.

2. Effector Cells:

Effector cells, which include clones, cell line, and PBMC samples were frozen and analyzed in batches. The cells were thawed the day before an experiment for overnight culture in CTL media. The following morning, viable cells were isolated by ficoll density centrifugation, washed, and resuspended to the appropriate concentration (usually 10⁷/ml) in CTL media.

3. Experimental Procedure:

All assays were done at least twice with duplicates for each condition. The optimum conditions for the assay were determined by extensive titrations of incubation times, effector:target ratios, antibodies concentration, and staining conditions (Betts, M. et al. Sensitive and viable identification of antigen-specific CD8+ T cells by a flow cytometric assay for degranulation. J Imm Methods in press(2003)) (unpublished data). The effector:target ratio used was generally 1:2, with 2×10⁵ for clones or 10⁶ for the cell line and patient PBMC samples. To each well in a flexible 96 well plate, the following were added in order: 1 μl of 2 mM monensin (Sigma) in 100% EtOH, 100 μl target cells, 100 μl of effector cells, and 1 μl of anti-human CD107a-APC antibodies. The cells were mixed well using a multichannel pippetor. The plate was centrifuged at 300×g for 1 min to pellet cells, then placed into an incubator at 37° C. for 5 hours. After the incubation, the plates were centrifuged to 500×g to pellet cells and the supernatant was removed. Cell-cell conjugates were disrupted by washing the cells with PBS supplemented with 0.02% azide and 0.5 mM EDTA, and mixed vigorously using a multichannel pippetor. Cells were washed twice and stained with additional antibodies.

E. Flow Cytometry Analysis

Cells were stained with anti-human CD3-FITC (Caltag), CD8-PE (Caltag) and CD19-CyChrome (BD Biosciences) antibodies. The final staining dilution of each antibody was 1/20, 1/600 and 1/80, respectively. Alternatively, cells were stained with anti-human CD8-FITC (Caltag), tetramer-PE (Immunomics), and CD19-CyChrome. Cells were incubated on ice for 30 mins, washed, then analyzed using a two-laser, 4-color FACSCalibur (Becton Dickinson, San Jose, Calif.). At least one million events were acquired and analyzed using FlowJo (TreeStar, San Carlos, Calif.). Lymphocytes were identified by forward and side scatter signals, then selected for CD8+ and CD19−. Gated cells were plotted for CD107a versus CD3 (or tetramer) to determine the fraction of CD3+, CD8+, CD19− cells that was CD107a+. Intracellular staining of T cell clones for granule expression was done with Granzyme A-FITC (Pharmingen), anti-human perforin-PE (Pharmingen), anti-human CD8-PerCP5.5 (BD Biosciences), and Granzyme B-APC (Pharmingen) antibodies, using the Cytofix/Cytoperm kit (BD Biosciences).

II. Results

A. Relationship Between T Cell Recognition Efficiency, CD107a Mobilization, and Tumor Cytotoxicity

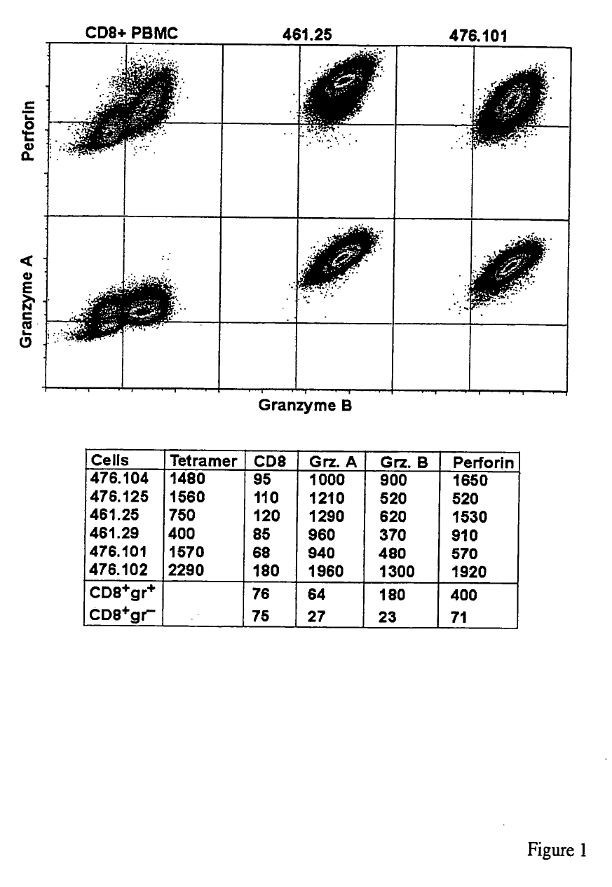

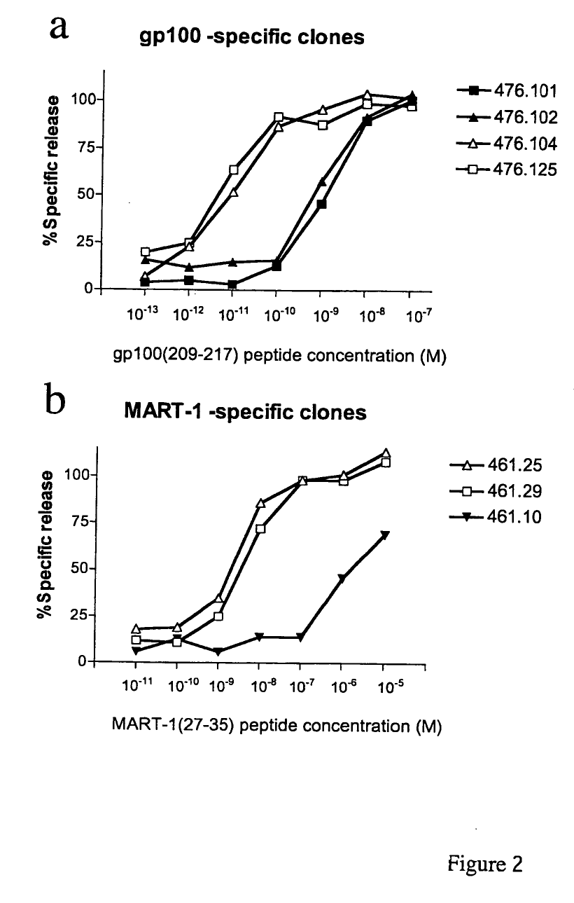

MART− or gp100-specific CD8+ T cell clones were generated from HLA-A*0201 (A2+) melanoma patients vaccinated with the TAAs MART 26-35 (27L) and gp100 209-217 (G209-2M) peptides. Antigen-specificity of these clones was confirmed by tetramer staining. These clones were indistinguishable in terms of CD8 expression or intensity of tetramer staining for these peptides (FIG. 1). However, when the relative recognition efficiency of each clone for the cognate native peptide was determined by peptide titration using a standard chromium release assay, they were found to be significantly different (FIGS. 2a and 2b). In addition, each clone was tested for cytolytic activity against three melanoma targets: mel526 (A2+, MART+, gp100+), Malme-3M (A2+, MART+, gp100+), and A375 (A2+, MART−, gp100−). Clones which were cytolytic for melanoma cell lines in an antigen-specific manner (positive for mel526 and Malme-3M, and negative for A375) were consistently found to be of high recognition efficiency (10⁻¹⁰ to 10⁻¹² M). Those that did not kill melanoma cells were of low recognition efficiency (10⁻⁸ to 10⁻⁹ M). These data are summarized in Table 1.

TABLE 1
Cytolytic activity against tumor targets and granule expression
of high and low recognition efficiency (RE) TAA-specific T
cell clones. High RE (476.104, 476.125, 461.25, 461.29) and
low RE (476.101, 476.102) gp100-specific T cell clones were
incubated with ⁵¹Cr-labelled melanoma targets at E:T ratios
of 10:1. Each combination was done in triplicates and values
given are the average percent specific lysis ± SD. This
assay was done twice with similar results.

| Clone | Specificity | Malme3M | mel526 | A375 |
|---|---|---|---|---|
| 476.104 | gp100 | 59 ± 1 | 70 ± 8 | −1 ± 1 |
| 476.125 | gp100 | 60 ± 4 | 67 ± 9 | −1 ± 2 |
| 461.25 | MART | 52 ± 0 | 70 ± 6 | −1 ± 4 |
| 461.29 | MART | 52 ± 4 | 61 ± 1 | −1 ± 4 |
| 476.101 | gp100 | −1 ± 1 | −1 ± 1 | 2 ± 1 |
| 476.102 | gp100 | 1 ± 1 | 0 ± 1 | 1 ± 1 |

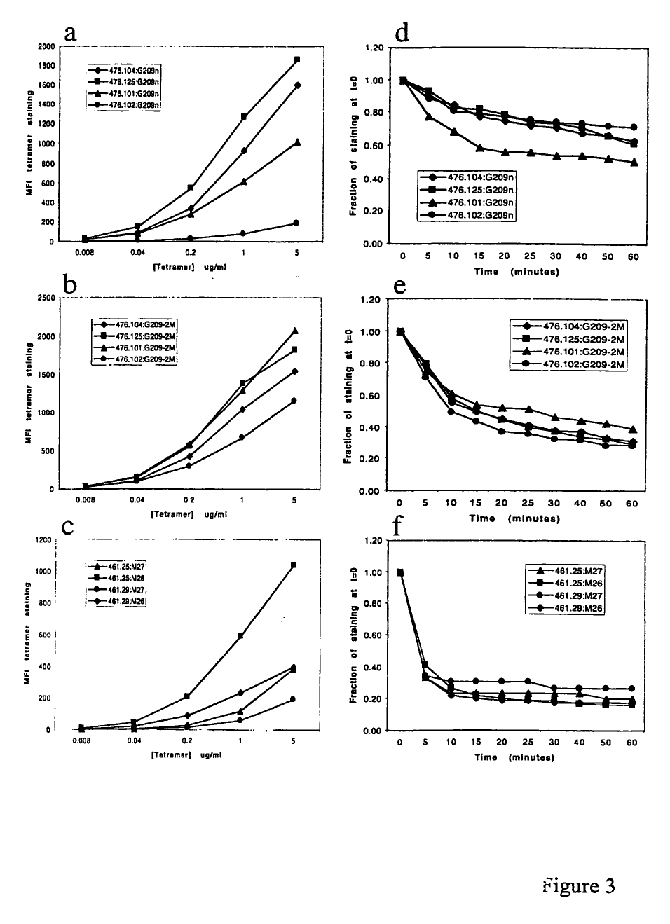

Differences in tumor cytolytic activity could not be explained by TCR, CD8, or granule expression (FIG. 1). To further investigate whether these recognition efficiency differences stem from differences in ‘structural avidity’ of these clones, we performed tetramer titrations and dissociation assays (FIGS. 3a to 3f). Tetramer titrations did suggest a difference in ‘structural avidity’ with regard to the G209 native peptide between the high and low recognition efficiency (RE) clones (FIG. 3a). However, these clones showed very similar binding to the heteroclitic G209-2M tetramers (FIG. 3b), demonstrating differential recognition of the two variant peptides by these T cells. Importantly, while the tetramer dissociation assays revealed a somewhat higher rate of dissociation with the G209 native tetramer for one low RE clone 476.101, the other low RE clone (476.102) showed no difference from the high RE clones (FIG. 3d). These data contrast with the clear-cut differences in peptide-reactivity of the high versus low RE clones by cytotoxicity assays (FIGS. 2a and 2b). In general, the MART-specific clones (461.25 and 461.29) exhibited lower peptide reactivity, tetramer staining, and faster tetramer dissociations (for native and heteroclitic peptides) even though these clones were both tumor-cytolytic.

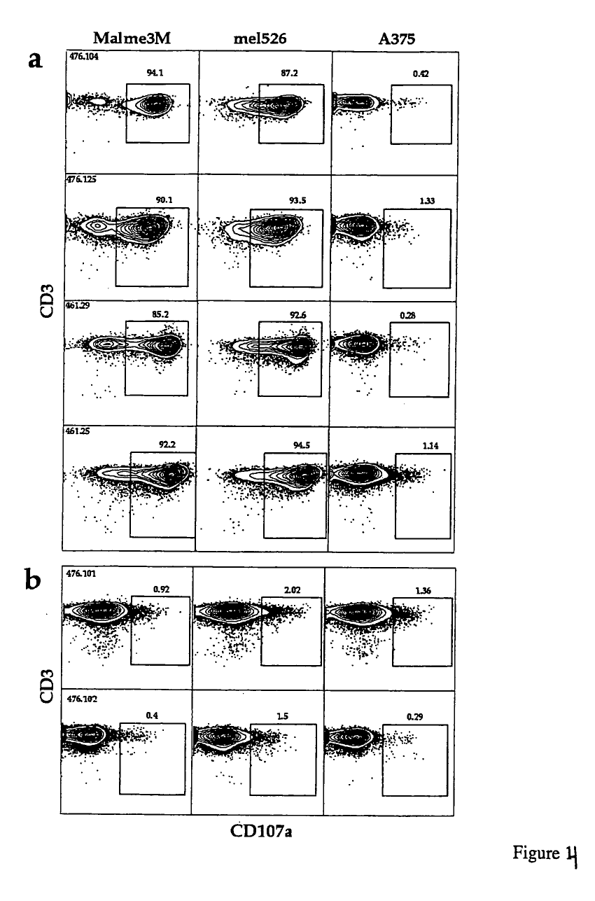

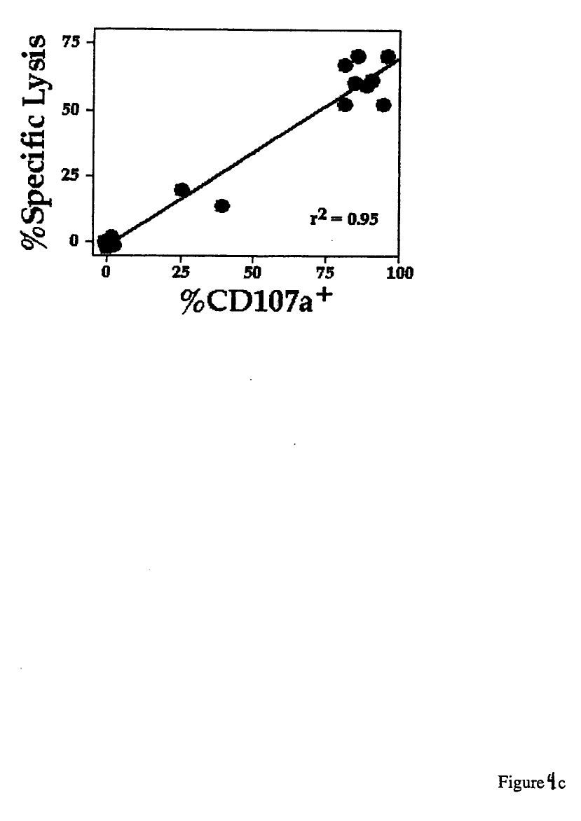

Clones of high and low recognition efficiency were selected for analysis by CD107a surface expression. Incubation of T cell clones of different functional avidities with tumor targets revealed specific yet different abilities to mobilize CD107a. Four high RE clones (two MART-specific and two gp100-specific) were incubated with mel526, Malme-3M, or A375 at a 1:1 ratio for 5 hours at 37° C. Anti-CD107a antibodies were present during the incubation period; following incubation, cells were stained with additional antibodies and analyzed by flow cytometry. All four high RE clones mobilized CD107a in an antigen-specific manner—i.e., positive for mel526 and Malme-3M, compared to ˜1% CD107a positive for A375 (FIG. 4a). In contrast, low RE clones did not mobilize CD107a after exposure to mel526, Malme-3M or A375 (FIG. 4b). To correlate CD107a mobilization with cytolytic activity, cytotoxicity data generated using ⁵¹Cr release assay for each of the four clones were plotted against corresponding CD107a mobilization (FIG. 4c). The r² of 0.94 reflects a strong correlation between CD107a mobilization and target lysis by these effectors.

B. Tumor Reactive T Cells Identified from a Heterogeneous Cell Line

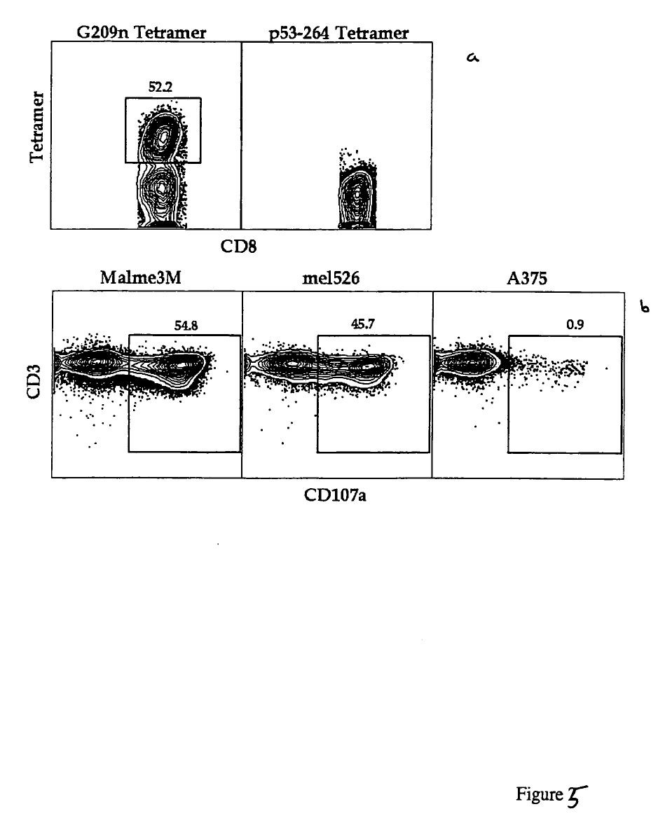

To establish that the CD107a flow cytometric assay could be used to identify tumor-reactive cells from a heterogeneous population, we generated a T cell line enriched for gp100-reactivity. PBMCs from a melanoma patient vaccinated with gp100-210M (G209-2M) were repeatedly stimulated with the native gp100 209-217 peptide (G209n) in vitro in the presence of low dose IL-2. After three weeks, the resulting cell line was stained with pMHC tetramers made with the native gp100 peptide and analyzed by flow cytometry. This CTL line was found to be 52% G209n-specific by tetramer staining (FIG. 5a). To determine if these gp100 tetramer+ cells could be identified using the CD107a assay, we incubated the CTL line with mel526, Malme-3M, and A375 as above. About 50% of CD8+ T cells in the line mobilized CD107a in response to Malme-3M and mel526, but not to A375 (FIG. 5b). This correlation between percent CD107a+ and percent tetramer+ cells upon mel526 and Malme-3M stimulation suggests that the T cells in this line elicited by repeated stimulations with G209n were indeed of high recognition efficiency and tumor-reactive.

C. Tumor Reactive T Cells Identified from Post-Vaccine PBMC

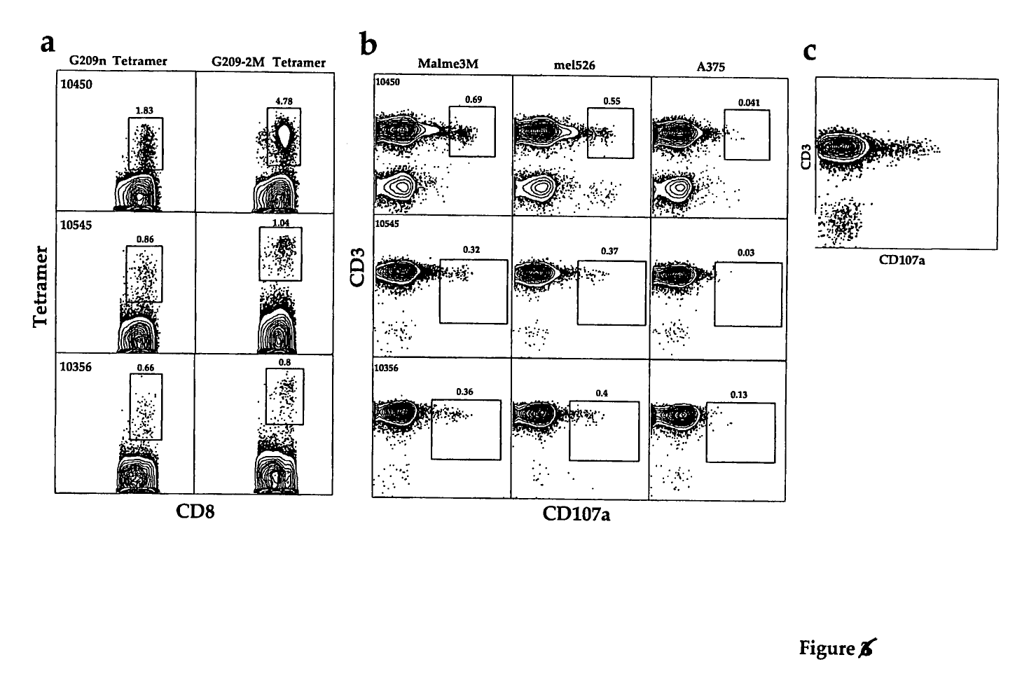

We further sought to determine whether we could identify rare tumor-reactive T cell populations directly from patient PBMCs. Three post-vaccination PBMC samples containing gp100 tetramer+ T cells were analyzed by staining with CD107a antibodies during the stimulation, followed by staining with other antibodies and analysis by flow cytometry. Flow cytometric analysis of these samples with HLA-A*0201 tetramers made with either the native gp100 or G209-2M peptide are shown in FIG. 6a. Tetramer analysis showed that the patients responded to the G209-2M peptide vaccine with an increase from less than 1 in 10,000 CD8+ T cells to 4.8%, 0.8%, and 1.0% tetramer+ cells for 10450, 10356, and 10545, respectively. However, staining with tetramers made with the gp100 native peptide consistently yielded smaller populations than with G209-2M heteroclitic tetramers—1.8%, 0.66%, and 0.86% for 10450, 10356, and 10545 respectively—suggesting that not all of the vaccine-induced T cells were specific for the native gp100 peptide and hence potentially capable of killing tumor. To address this issue, we analyzed these samples for CD107a mobilization upon stimulation with melanoma targets. As shown in FIG. 6b, small but clear populations of CD3+ CD8+ T cells mobilized CD107a specifically to Malme-3M and mel526 (but not A375) in all three post-vaccine samples tested. The fractions of CD8+ CD107a+ cells were approximately 0.8% for 10450, 0.25% for 10356, and 0.3% for 10545 (averages of two independent experiments). These data suggest that of peptide-specific T cells elicited by vaccination with the heteroclitic peptide G209-2M, only a fraction are specific for the native gp100 209-217 peptide, and only a fraction of these may be truly tumor-reactive.

D. Functional Analysis of CD107a+ Cells

To confirm that T cells which mobilize CD107a expression after tumor stimulation were indeed tumor-reactive, we cloned and analyzed CD107a+ cells from PBMC sample 10450 after incubation with Malme-3M. FIG. 6c shows the gates used to isolate cells for cloning. Six clones each from the CD107a+ and CD107a− gates were expanded and analyzed for cytotoxicity and recognition efficiency. To confirm antigen-specificity, we stained these clones with G209-2M tetramers and found that all CD107a+ clones were G209-specific but not the CD107a− clones (data not shown). As shown in Table 2, the CD107a+ clones were found to be cytolytic against mel526 and Malme-3M (and not A375) in chromium release assays, while the CD107a− clones were not (p<0.001). Furthermore, CD107a+ clones were analyzed for recognition efficiency by peptide titration and confirmed to be of high recognition efficiency (10⁻¹⁰ to 10⁻¹² M).

Table 2. Cytolytic activity and recognition efficiency of CD107a+ and CD107a− clones. CD107a+ and CD107a− clones were generated from vaccinated patient sample 10450 from flow cytometrically-sorted cells using analysis such as that shown in FIG. 3C. Six CD107a+ and six CD107a− clones were selected for cytotoxicity analysis against Malme-3M at E:T ratios of 10:1. The values given are averages of triplicate readings. The averages of the six CD107a+ or CD107a− clones are shown on the bottom row. The six CD107a+ clones were further analyzed for recognition efficiency for G209n by peptide titration as described in materials and methods. Data is representative of two independent experiments.

| | |
|---|---|
| Average % cytotoxicity | Recognition efficiency (M) |

| CD107a+ | CD107a− | CD107a+ |
|---|---|---|
| 45 | −2 | 10⁻¹² |
| 13 | −3 | 10⁻¹¹ |
| 42 | −3 | 10⁻¹¹ |
| 35 | −2 | 10⁻¹¹ |
| 46 | −5 | 10⁻¹² |
| 31 | −5 | 10⁻¹¹ |
| 35.3 | −3.3 | |

E. Combination of CD107a Mobilization with Tetramer Staining

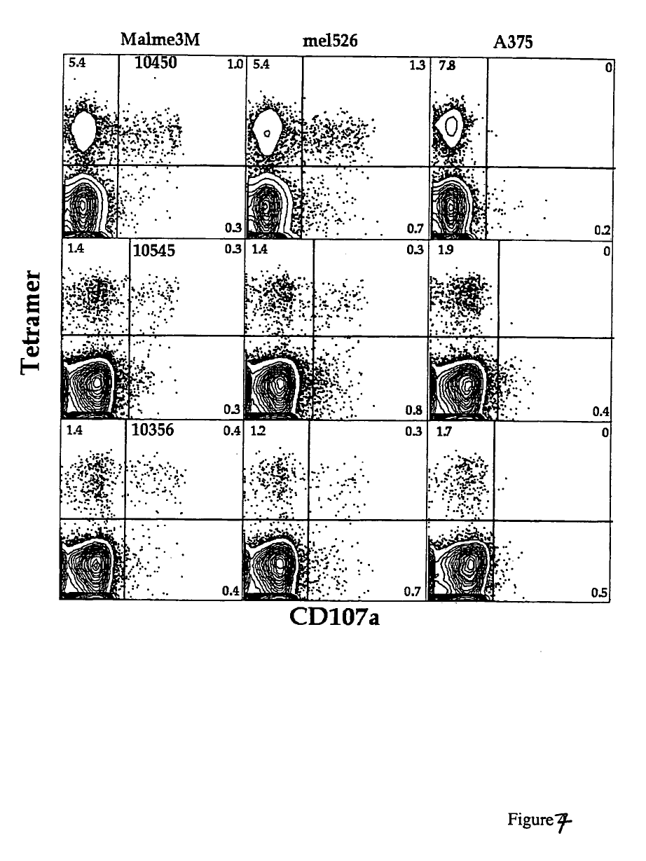

To directly assess the proportion of tetramer+ cells which are of high recognition efficiency and tumor-reactive, CD107a exposure was combined with tetramer staining. Patient PBMC samples were incubated with target cells (in the presence of anti-CD107a antibodies) for 5 hours, then stained with tetramers, anti-CD8 and anti-CD19 antibodies, and analyzed by FACS. Lymphocytes were identified based on forward and side scatter, and CD8 T cells were further identified as CD8+ and CD19−. Finally, CD107a was plotted versus tetramer staining. As shown in FIG. 7, tetramer+ events segregated into CD107a+ and CD107a− subsets. The proportion of tetramer+ cells which mobilized CD107a upon stimulation with melanoma targets Malme-3M and mel526 was remarkably consistent amongst all three samples, in the 10-20% range (Table 3). This result indicates that high recognition efficiency, tumor-reactive cells represent a small subset of peptide-specific T cells elicited by vaccination in these patients.

Table 3. Average percentages of G209-2M tetramer+ cells mobilizing CD107a upon stimulation with tumor targets. Patient samples were incubated with indicated melanoma target cells and fractions of G209-2M tetramer+ cells which upregulated CD107a was determined. Values given are the average ±SD of 4-6 independent measurements.

| Patient | | | |
|---|---|---|---|
| sample | Malme-3M | Mel526 | A375 |
| 10450 | 16.7 ± 1.0 | 16.4 ± 2.1 | 0.2 ± 0.3 |
| 10545 | 15.7 ± 1.7 | 17.1 ± 1.4 | 0.1 ± 0.1 |
| 10356 | 16.5 ± 4.3 | 15.0 ± 4.4 | 0.1 ± 0.1 |

Tetramer+ CD107a+ and tetramer+ CD107a− T cells were sorted independently from patient samples 10545 and 10356. Five to seven tetramer+ CD107a− and tetramer+ CD107a+ clones from each sample were expanded and analyzed for cytolytic activity against tumor targets. As shown in Table 4, there were significant differences in cytolytic activity between tetramer+ CD107a+ and tetramer+ CD107a− clones against the melanoma targets Malme-3M and mel526.

Table 4. Cytolytic activity of tetramer+ CD107a+ and tetramer+ CD107a− clones. Tetramer+ CD107a+ and tetramer+ CD107a− clones were generated from vaccinated patient samples 10545 (A) and 10356 (B) via FACSorting using gates shown in FIG. 4. Five to seven tetramer+ CD107a+ and tetramer+ CD107a− clones from each sort were selected for cytotoxicity analysis against melanoma targets Malme-3M, mel526, and A375 at E:T ratios of 10:1. The values given (percentage lysis) are averages of triplicate readings. The averages of the cytotoxicity results from the CD107a+ or CD107a− clones are shown on the bottom row, and are statistically different between CD107a+ and CD107a− clones against both melanoma targets Malme and mel526 (p<0.01 for 10545, p=0.03 for 10356). These clones were further analyzed for their recognition efficiency (RE) for G209n by peptide titration on T2 targets as described in materials and methods. Data is representative of two independent experiments.

A. Sample 10545

| Tetramer+ CD107a+ clones | | | | Tetramer+ CD107a− clones | | | |
|---|---|---|---|---|---|---|---|
| Malme | mel526 | A375 | RE | Malme | mel526 | A375 | RE |
| 31 | 38 | −1 | 10⁻¹² M | 0 | 0 | 0 | 10⁻⁸ M |
| 20 | 22 | −1 | 10⁻¹⁰ M | 8 | 4 | −1 | 10⁻⁹ M |
| 20 | 22 | −1 | 10⁻¹¹ M | 11 | 7 | 0 | 10⁻⁹ M |
| 27 | 29 | −1 | 10⁻¹¹ M | 24 | 20 | 1 | 10⁻¹⁰ M |
| 20 | 15 | −1 | 10⁻¹¹ M | 2 | 1 | 0 | 10⁻⁸ M |
| | | | | 4 | 8 | −1 | 10⁻⁹ M |
| | | | | 1 | 5 | −1 | 10⁻⁸ M |
| 23.6 | 25.2 | −1.0 | | 7.1 | 6.4 | −0.3 | |

B. Sample 10356

| Tetramer+ CD107a+ clones | | | | Tetramer+ CD107a− clones | | | |
|---|---|---|---|---|---|---|---|
| Malme | mel526 | A375 | RE | Malme | mel526 | A375 | RE |
| 40 | 42 | 1 | 10⁻¹¹ M | 2 | 6 | 0 | 10⁻⁸ M |
| 37 | 32 | 2 | 10⁻¹¹ M | 2 | 5 | 0 | 10⁻⁷ M |
| 40 | 42 | 3 | 10⁻¹¹ M | 1 | 3 | 1 | 10⁻⁷ M |
| 33 | 32 | 3 | 10⁻¹⁰ M | 42 | 47 | 0 | 10⁻¹¹ M |
| 32 | 34 | 2 | 10⁻¹⁰ M | 2 | 5 | 1 | 10⁻⁹ M |
| 39 | 51 | 1 | 10⁻¹² M | | | | |
| 36.8 | 38.8 | 2.0 | | 9.8 | 13.2 | 0.4 | |

As predicted, all tetramer+ CD107a+ clones were cytolytic for the relevant tumor targets, and most tetramer+ CD107a− clones were not. All clones efficiently lysed T2 cells pulsed with >100 ng/ml peptides (>50% lysis), suggesting that differences in their tumor reactivity did not stem from dysfunction of certain clones. Interestingly, one of seven tetramer+ CD107a− clones from 10545 and one of five tetramer+ CD107a− clones from 10356 exhibited specific cytolytic activity against Malme-3M and mel526, and not A375. These clones were further analyzed for recognition efficiency for the G209n peptide and confirmed that tetramer+ CD107a+ clones were of high recognition efficiency (10⁻¹⁰ to 10⁻¹² M), while all but two tetramer+ CD107a− clones were of low recognition efficiency (10⁻⁸ to 10⁻⁹ M). The exceptions were the two clones which exhibited specific cytolytic activity against melanoma targets, with functional avidities of 10⁻¹⁰ to 10⁻¹¹ M.

III. Discussion

The above results show that the identification of cells which mobilize CD107a to the cell surface following stimulation is an excellent measure of cytolytic capacity. The above results also show that detection of the mobilization of CD107a upon interaction with tumor targets also identifies T cells of high recognition efficiency.

Based on the above results, it is important to make a distinction between recognition efficiency, functional capacity, and cytolytic potential against tumor of a T cell. All six clones presented in Table 1 were of a functional state (not anergic) as they were all capable of lysing T2 targets pulsed with sufficient relevant peptides. This was in fact how we measured (by definition) the recognition efficiency of each clone. However, only clones of high recognition efficiency for the TMs MART or gp100 degranulated upon melanoma stimulation (by CD107a exposure) and lysed melanoma targets on cytotoxicity assays. Hence, our data demonstrate that peptide-specificity does not necessarily equate to tumor-reactivity—recognition efficiency is a critical factor. Our results show a correlation between recognition efficiency and tumor cytolytic potential, which is distinct from functional capacity. Moreover, tumor cytolytic potential is not merely a reflection of cytolytic granules expression, as some clones which expressed high levels of perforin and granzyme did not degranulate or kill melanoma targets (FIG. 1). These data highlight the fact that killing is a decision by a T cell based on the aggregate of its input from the target cell and recognition efficiency. Surface mobilization of CD107 to tumor stimulation is a measure of degranulation and is the first assay which directly measures tumor-reactivity in a rapid and reliable fashion.

The ability to use established melanoma lines mel526 and Malme-3M as tumor targets (for HLA-A*0201 patients) represents a significant advantage over having to use autologous tumor targets for each patient. Primary melanoma cell lines from patient samples are difficult and laborious to establish, and ultimately successful in only a proportion of patients. While mel526 and Malme-3M are HLA-A2+, there would almost certainly be mismatches for other HLA alleles leading to the possibility of alloreactivity. However, we did not observe a significant level of non-specific killing or CD107a exposure due to alloreactivity. This may be due to lower recognition efficiency of alloreactive T cells than of the desired tumor-reactive T cells—as we demonstrated, only high recognition efficiency T cells mobilizedCD107 after stimulation with specific targets. In addition, the 5-hour period in which this assay is performed may be insufficient for the elicitation of most alloreactive T cells.

CD107a mobilization may be combined with tetramer staining to directly assess the functional capacity of peptide-specific T cells. As shown in FIG. 6, the percentage of cells staining with the G209 native tetramer was consistently lower than those staining with the G209-2M tetramer in patients vaccinated with the G209-2M peptide. This finding indicates that a proportion of G209-2M-specific T cells cross-react with the native G209 peptide with sufficient avidity to stain with the G209n tetramer. This would have important clinical implications since tumor cells express only the native peptide, and at very low concentrations on the cell surface. Furthermore, the CD107a assay showed that the proportion of T cells capable of mobilizing CD107a represents an even smaller fraction (30-50%) of the cells staining with the G209n tetramer. Thus, even for G209n-specific T cells, only a subset is of sufficient avidity or in a functional state to kill tumor targets. This was confirmed by the combination of tetramer staining with CD107a (FIG. 7 and Table 3), demonstrating that only 10-20% of G209-2M tetramer+ cells degranulated in response to melanoma. In contrast, >80% of CMV-specific T cells degranulate in response to cognate peptide stimulation (Rubio and Lee, unpublished data).

A significant difference in function between tetramer+ CD107a+ and tetramer+ CD107a− cells was confirmed by sorting and cloning such cells independently. As shown in Table 4, there is a statistically significant difference in cytolytic activity against tumor targets between tetramer+ cells that could mobilizeCD107a and those that could not. As predicted, tetramer+ CD107a+ clones were tumor cytolytic and of high recognition efficiency. Interestingly, while most tetramer+ CD107a− were non-tumor cytolytic and of low recognition efficiency, one clone from each sample exhibited specific cytolytic activity to tumor. These clones may represent cells that are of intermediate RE or functionality in what is likely a continuous distribution of cytolytic potential of effector cells. Alternatively, the parental cells for these clones might have been anergic in vivo (Lee, P. P. et al. Characterization of circulating T cells specific for tumor-associated antigens in melanoma patients. Nature Medicine 5, 677-85 (1999)) and became reactivated upon in vitro stimulation and expansion. We are currently studying this issue in more detail.

A key advantage of the CD107 technique is the ability to detect tumor-reactive CD8+ T cells without knowing the peptide-MHC target. Since the assay measures T cells which degranulate in response to tumor cells, there is no a priori need to know the actual peptide target which would be required for most current assays. This is an important advantage since only a small number of tumor-associated antigens (TAAs) have been identified to-date, mostly in the setting of melanoma. In FIG. 7, cells that are CD107a+ tetramer− may represent possible candidates for tumor-reactive T cells not elicited by the vaccine (i.e., not gp100-specific). This technique may also be useful for immune monitoring of clinical trials involving vaccination with whole tumor cells, tumor-APC fusions, APCs pulsed with tumor lysates or transfected with tumor RNA, or other novel immunotherapeutic strategies in which the exact peptide targets are undefined. In such instances, the same cells used for vaccination could be used as stimulators in the immune monitoring assay to reveal tumor-reactive, cytolytic T cells.

To our knowledge, the above represents the first successful isolation of pure, viable populations of cytolytic tumor-reactive T cells directly from patient blood samples. We used flow cytometric quantification of the surface mobilization of CD107a—an integral membrane protein within cytolytic granules of cytotoxic T cells—as a marker for degranulation upon tumor stimulation. Mobilization of CD107a selectively identified T cells that were tumor-cytolytic. Using this technique, we show that tumor-reactive T cells are indeed elicited in patients post-cancer vaccination, and that tumor-reactivity is strongly correlated with recognition efficiency of the T cells for peptide-bearing targets. Combining CD107a mobilization with peptide/MHC tetramer staining, we directly correlated antigen-specificity and cytolytic ability on a single-cell level to show that high recognition efficiency, tumor-reactive T cells represent only a minority of peptide-specific T cells elicited in patients after heteroclitic peptide vaccination. These data strongly point to the importance of recognition efficiency of peptide-specific T cells in the design of future vaccination strategies. Moreover, we used flow cytometric sorting to directly isolate tumor-reactive T cells, and then expanded these cells ex vivo to high numbers. These techniques will be useful not only for immune monitoring of cancer vaccine trials, but also for adoptive cellular immunotherapy following ex vivo expansion. As such, we have developed a method which utilizes CD107a mobilization to identify and isolate functional, high recognition efficiency, tumor-reactive T cells directly from peripheral blood mononuclear cells (PBMC) of cancer patients post-vaccination. These data represent (to our knowledge) the first successful isolation of viable T cells based on a measurement of their capability to kill tumor targets.

In summary, we demonstrate that the granule membrane protein, e.g., CD107a, mobilization assay can be used to identify and viably isolate rare high recognition efficiency, tumor-reactive T cells from patient specimens. The ability to link antigen specificity with function, and to isolate such cells by sorting, will make this technique useful in immune monitoring and adoptive cellular immunotherapy for cancer. Furthermore, these data strongly point to the importance of recognition efficiency in the design of future vaccination and immunotherapeutic strategies.

It is apparent from the above results and discussion that the subject invention provides convenient protocols for isolating high recognition efficiency cytolytic cells from a sample. Because target cell stimulators that endogenously express target peptides are employed in the subject methods, as opposed to cells pulsed with target peptides, the methods identify cytolytic cells that have high recognition efficieny for naturally occurring target cells. Accordingly, the subject invention is capable of identifying/isolating cells that are truly cytolytic for a target cell as it naturally occurs, and not just a cell pulsed with the target peptide. As such, the subject invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for assaying a sample for a lymphocyte cytolytic for a target cell, said method comprising:
combining said sample with a target cell stimulator and a detectably labeled granule membrane protein specific binding agent, wherein said target cell stimulator is a cell or derivative thereof that endogenously expresses a target peptide of interest; and
identifying any resultant lymphocytes labeled with said granule membrane protein specific binding agent as cytolytic for said target cell.

2. The method according to claim 1, wherein said cytolytic lymphocyte is a T-cell.

3. The method according to claim 1, wherein said target cell stimulator is a cell.

4. The method according to claim 1, wherein said granule membrane protein is chosen from CD107a, CD107b, CD63, CTLA-4, Man-6-PR and TIA/GMP-17.

5. The method according to claim 1, wherein said method further comprises contacting said sample with a detectably labeled T-cell specific binding agent.

6. The method according to claim 5, wherein said T-cell specific binding agent specifically binds to CD3.

7. The method according to claim 1, wherein said method further comprises contacting said sample with a detectably labeled cytotoxic T-cell specific binding agent.

8. The method according to claim 7, wherein said cytotoxic T-cell specific binding agent specifically binds to CD8.

9. The method according to claim 1, wherein said detectably labeled binding agent is labeled with a fluorescent label.

10. The method according to claim 9, wherein any resultant T-cells labeled with said granule membrane protein specific binding agentare identified flow cytometrically.

11. The method according to claim 1, wherein said method further comprises separating any resultant lymphocytes labeled with said granule membrane protein specific binding agent from other components of said sample to produce a composition enriched for lymphocytes cytolytic for said target cell.

12. The method according to claim 1, wherein said sample is a blood sample.

13. The method according to claim 12, wherein said blood sample is a peripheral blood mononuclear cell sample.

14. The method according to claim 1, wherein said sample is from a subject vaccinated with an immunogen for said target cell.

15. A method of identifying the presence of a lymphocyte cytolytic for a target cell in a subject, said method comprising:
assaying a sample from said subject for a lymphocyte cytolytic for said target cell according to the method of claim 1 to identify said lymphocyte cytolytic for said target cell.

16. The method according to claim 15, wherein said assaying is performed at least two different times in order to monitor said subject for the presence of said lymphocyte cytolytic for said target cell.

17. The method according to claim 16, wherein said method is a method of monitoring said subject for progression of a disease condition.

18. The method according to claim 17, wherein said disease condition is a neoplastic disease condition.

19. A method for treating a subject for a target cell mediated disease condition, said method comprising:
obtaining a composition enriched for a population of lymphocytes cytolytic for said target cell according to the method of claim 11;
expanding said population of lymnphocytes in said composition; and
administering said expanded population of lymphocytes to said subject.

20. The method according to claim 19, wherein said target cell mediated disease condition is a neoplastic disease condition.

21. The method according to claim 19, wherein said target cell mediated disease condition is a viral disease condition.

22. A substantially pure composition of viable lymphocyates cytolytic for a target cell.

23-25. (canceled)

26. A kit for use in a method according to claim 1, said kit comprising:
(a) a detectably labeled specific binding agent that specifically binds to a granule membrane protein; and
(b) instructions for using said binding agent in a method according to claim 1.

27-34. (canceled)

35. A labeled sample comprising:
(a) a sample medium;
(b) a detectably labeled granule membrane protein specific binding agent; and
(c) a detectably labeled T-cell specific binding agent.

36. (canceled)
